(19) United States
(12) Patent Application Publication
(10) Pub. No.: US 2006/0235012 A1
(43) Pub. Date: Oct. 19, 2006

(54) Carboline and betacarboline derivatives for use as hdac enzyme inhibitors

(75) Inventors: Alan Hornsby Davidson, Oxfordshire (GB); Christopher John Yarnold, Oxon (GB); Charlton Michael Hugh, Oxon (GB)

Correspondence Address: BANNER & WITCOFF, 1001 G STREET N W, SUITE 1100, WASHINGTON, DC, 20001, US

(73) Assignee: Chroma Therapeutics Limited, Oxfordshir (GB)

(21) Appl. No.: 10/559,626

(22) PCT Filed: Jun. 15, 2004
(86) PCT No.: PCT/GB04/02504

§ 371 Date: Jun. 7, 2006

(30) Foreign Application Priority Data
Jun. 16, 2003 (GB) 0313814.6
Dec. 23, 2003 (GB) 0329998.9

(51) Int. Cl. A61K 31/5377; A61K 31/506; A61K 31/4745; C07D 471/02

(52) U.S. Cl. 514/234.2; 514/291; 546/85; 514/256; 544/127; 544/294

(57) ABSTRACT

Compounds of formula (IA) and (IB) are inhibitors of histone deacetylase activity and useful for the treatment of, inter alia, cancers: wherein fused rings A¹ and A² are optionally substituted; linker radical R₁ represents a radical of formula

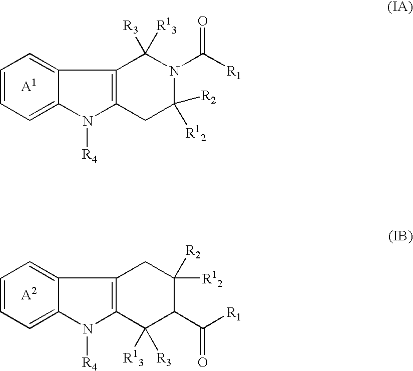

This invention relates to compounds which inhibit members of the histone deacetylase family of enzymes and to their use in the treatment of cell proliferative diseases, including cancers, polyglutamine diseases for example Huntingdon disease, neurogenerative diseases for example Alzheimer disease, autoimmune disease and organ transplant rejection, diabetes, haematological disorders and infection.

### BACKGROUND TO THE INVENTION

In eukaryotic cells DNA is packaged with histones, to form chromatin. Approximately 150 base pairs of DNA are wrapped twice around an octamer of histones (two each of histones 2A, 2B, 3 and 4) to form a nucleosome, the basic unit of chromatin. The ordered structure of chromatin needs to be modified in order to allow transcription of the associated genes. Transcriptional regulation is key to differentiation, proliferation and apoptosis, and is, therefore, tightly controlled. Control of the changes in chromatin structure (and hence of transcription) is mediated by covalent modifications to histones, most notably of the N-terminal tails. Covalent modifications (for example methylation, acetylation, phosphorylation and ubiquitination) of the side chains of amino acids are enzymatically mediated (A review of the covalent modifications of histones and their role in transcriptional regulation can be found in Berger S L 2001 Oncogene 20, 3007-3013; See Grunstein, M 1997 Nature 389, 349-352; Wolffe A P 1996 Science 272, 371-372; and Wade P A et al 1997 Trends Biochem Sci 22, 128-132 for reviews of histone acetylation and transcription).

Acetylation of histones is associated with areas of chromatin that are transcriptionally active, whereas nucleosomes with low acetylation levels are, typically, transcriptionally silent. The acetylation status of histones is controlled by two enzyme classes of opposing activities; histone acetyltransferases (HATs) and histone deacetylases (HDACs). In transformed cells it is believed that inappropriate expression of HDACs results in silencing of tumour suppressor genes (For a review of the potential roles of HDACs in tumorigenesis see Gray S G and Teh B T 2001 Curr Mol Med 1, 401-429).

Inhibitors of HDAC enzymes have been described in the literature and shown to induce transcriptional reactivation of certain genes resulting in the inhibition of cancer cell proliferation, induction of apoptosis and inhibition of tumour growth in animals (For review see Kelly, W K et al 2002 Expert Opin Investig Drugs 11, 1695-1713). Such findings suggest that HDAC inhibitors have therapeutic potential in the treatment of proliferative diseases such as cancer (Kramer, O H et al 2001 Trends Endocrinol 12, 294-300, Vigushin D M and Coombes R C 2002 Anticancer Drugs 13, 1-13).

In addition, others have proposed that aberrant HDAC activity or histone acetylation is implicated in the following diseases and disorders; polyglutamine disease, for example Huntingdon disease (Hughes R E 2002 Curr Biol 12, R141-R143; McCampbell A et al 2001 Proc Soc Natl Acad Sci 98, 15179-15184; Hockly E et al 2003 Proc Soc Natl Acad Sci 100, 2041-2046), other neurodegenerative diseases, for example Alzheimer disease (Hempen B and Brion J P 1996, J Neuropathol Exp Neurol 55, 964-972), autoimmune disease and organ transplant rejection (Skov S et al 2003 Blood 101, 1430-1438; Mishra N et al 2003 J Clin Invest 111, 539-552), diabetes (Mosley A L and Ozcan S 2003 J Biol Chem 278, 19660-19666) and diabetic complications, infection (including protozoal infection (Darkin-Rattray, S J et al 1996 Proc Soc Natl Acad Sci 93, 13143-13147)) and haematological disorders including thalassemia (Witt O et al 2003 Blood 101, 2001-2007). The observations contained in these manuscripts suggest that HDAC inhibition should have therapeutic benefit in these, and other related, diseases.

### BRIEF DESCRIPTION OF THE INVENTION

This invention is based on the finding that a class of tricyclic nitrogen-containing compounds having a hydroxamate or N-hydroxy acylamino metal binding group are capable of inhibiting the activity of members of the HDAC family, including HDAC1, and are of value in the treatment of diseases mediated by excessive or inappropriate HDAC, especially HDAC1 activity, such as cell-proliferative diseases, including cancers, polyglutamine diseases for example Huntingdon disease, neurogenerative diseases for example Alzheimer disease, autoimmune disease and organ transplant rejection, diabetes, haematological disorders and infection (including but not limited to protozoal and fungal).

### DETAILED DESCRIPTION OF THE INVENTION

In a broad aspect, the present invention provides a compound of formula (IA) or (IB), or a salt, hydrate or solvate thereof. 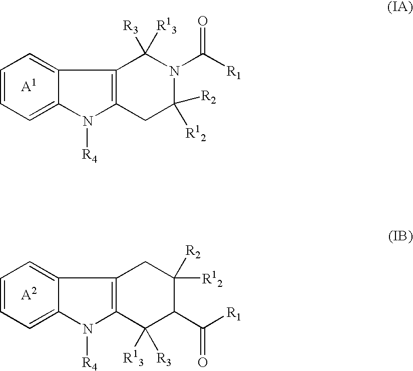
wherein
fused rings A¹ and A² are optionally substituted;
R₁ represents a radical of formula -(Alk¹)ₙ-(X)ₘ-(Alk²)ₚ-Z wherein
Z represents a radical of formula —C(═O)NH(OH), or —N(OH)C(═O)Y wherein Y represents hydrogen, C₁-C₆ alkyl, a phenyl or cycloalkyl ring, or a monocyclic heterocyclic radical having 5 or 6 ring atoms;
Alk¹ represents an optionally substituted, straight or branched, C₁-C₆ alkylene radical,
Alk² represents an optionally substituted, straight or branched, C₁-C₆ alkylene, C₂-C₆ alkenylene or C₂-C₆ alkynylene radical which may optionally contain an ether (—O—), thioether (—S—) or amino (—NR^{A}—) link wherein R^{A} is hydrogen or C₁-C₃ alkyl;
X represents an optionally substituted phenyl or 5- or 6-membered heteroaryl ring; and
n, m and p are independently 0 or 1, provided that at least one of n, m and p is 1 and the length of radical -(Alk¹)ₙ-(X)ₘ-(Alk²)ₚ- is equivalent to that of a hydrocarbon chain of from 2-10 carbon atoms;

R¹₂ is hydrogen and R₂ is (a) an optional substituent or (b) a radical of formula -(Alk³)ᵣ-Q wherein r is 0 or 1, Alk³ represents an optionally substituted, straight or branched, C₁-C₆ alkylene, C₂-C₆ alkenylene or C₂-C₆ alkynylene radical and Q is hydrogen or an optionally substituted carbocyclic or heterocyclic group; or R¹₂ and R₂ taken together with the carbon atoms to which they are attached form an optionally substituted carbocyclic or heterocyclic ring;

R¹₃ is hydrogen and R₃ is (i) an optional substituent or (ii) a radical of formula -(Alk³)ᵣ-Q wherein r is 0 or 1, Alk³ represents an optionally substituted, straight or branched, C₁-C₆ alkylene, C₂-C₆ alkenylene or C₂-C₆ alkynylene radical and Q is hydrogen or an optionally substituted carbocyclic or heterocyclic group; or R¹₃ and R₃ taken together with the carbon atoms to which they are attached form an optionally substituted carbocyclic or heterocyclic ring; and

R₄ is hydrogen or C₁-C₆ alkyl.

In another broad aspect the invention provides the use of a compound of formula (I) as defined above, or a salt, hydrate or solvate thereof in the preparation of a composition for inhibiting the activity of an HDAC enzyme.

The compounds with which the invention is concerned may be used for the inhibition of HDAC activity, particularly HDAC1 activity, ex vivo or in vivo.

In one aspect of the invention, the compounds of the invention may be used in the preparation of a composition for the treatment of cell-proliferation disease, for example cancer cell proliferation, polyglutamine diseases for example Huntingdon disease, neurogenerative diseases for example Alzheimer disease, autoimmune disease and organ transplant rejection, diabetes, haematological disorders and infection (including but not limited to protozoal and fungal).

In another aspect, the invention provides a method for the treatment of cell-proliferation disease, for example cancer cell proliferation, polyglutamine diseases for example Huntingdon disease, neurogenerative diseases for example Alzheimer disease, autoimmune disease and organ transplant rejection, diabetes, haematological disorders and infection (including but not limited to protozoal and fungal), which comprises administering to a subject suffering such disease an effective amount of a compound of formula (I) as defined above.

As used herein the term “(C₁-C₆)alkyl” means a straight or branched chain alkyl moiety having from 1 to 6 carbon atoms, including for example, methyl, ethyl, n-propyl, isopropyl, n-butyl, isobutyl, sec-butyl, t-butyl, n-pentyl and n-hexyl.

As used herein the term “(C₁-C₆)alkylene radical” means a divalent saturated hydrocarbon chain having from 1 to 6 carbon atoms.

As used herein the term “(C₂-C₆)alkenyl” means a straight or branched chain alkenyl moiety having from 2 to 6 carbon atoms having at least one double bond of either E or Z stereochemistry where applicable. The term includes, for example, vinyl, allyl, 1- and 2-butenyl and 2-methyl-2-propenyl.

As used herein the term “divalent (C₂-C₆)alkenylene radical” means a divalent hydrocarbon chain having from 2 to 6 carbon atoms, and at least one double bond.

As used herein the term “C₂-C₆ alkynyl” refers to straight chain or branched chain hydrocarbon groups having from two to six carbon atoms and having in addition one triple bond. This term would include for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the term “divalent (C₂-C₆)alkynylene radical” means a divalent hydrocarbon chain having from 2 to 6 carbon atoms, and at least one triple bond.

As used herein the term “cycloalkyl” refers to a saturated carbocyclic radical having from 3-8 carbon atoms and includes, for example, cyclopropyl, cyclobutyl, cyclopentyl, cyclohexyl, cycloheptyl and cyclooctyl.

As used herein the term “cycloalkenyl” refers to a carbocyclic radical having from 3-8 carbon atoms containing at least one double bond, and includes, for example, cyclopentenyl, cyclohexenyl, cycloheptenyl and cyclooctenyl.

As used herein the term “aryl” refers to a mono-, bi- or tri-cyclic carbocyclic aromatic radical. Illustrative of such radicals are phenyl, biphenyl and napthyl.

As used herein the term “carbocyclic” refers to a cyclic radical whose ring atoms are all carbon, and includes aryl, cycloalkyl and cycloalkenyl radicals.

As used herein the term “heteroaryl” refers to an aromatic radical containing one or more heteroatoms selected from S, N and O. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in particular means a non-aromatic radical containing one or more heteroatoms selected from S, N and O. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as used herein means substituted with at least one substituent for example, selected from (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro and chloro), trifluoromethyl, trifluoromethoxy, trifluoromethylsulfonyl, nitro, nitrile (—CN), oxo, phenyl, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl or (C₃-C₈) cycloalkyl group. As used herein the term “optional substituent” means one of the foregoing substituents.

As used herein the term “salt” includes base addition, acid addition and quaternary salts. Compounds of the invention which are acidic can form salts, including pharmaceutically or veterinarily acceptable salts, with bases such as alkali metal hydroxides, e.g. sodium and potassium hydroxides; alkaline earth metal hydroxides e.g. calcium, barium and magnesium hydroxides; with organic bases e.g. N-ethyl piperidine, dibenzylamine and the like. Those compounds (I) which are basic can form salts, including pharmaceutically or veterinarily acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic and p-toluene sulphonic acids and the like.

Some compounds of the invention contain one or more actual or potential chiral centres because of the presence of asymmetric carbon atoms. The presence of several asymmetric carbon atoms gives rise to a number of diastereoisomers with R or S stereochemistry at each chiral centre. The invention includes all such diastereoisomers and mixtures thereof.

The Group R₁

The group Z in R₁ is a hydroxamate group —(═O)NHOH or N-hydroxy-acylamino group —N(OH)C(═O)Y, which functions as a metal binding group, interacting with the metal ion at the active site of the HDAC enzyme. At present a hydroxamate group is preferred.

The radical -(Alk¹)ₙ-(X)ₘ-(Alk²)ₚ- in R₁ functions as a linker radical, the length of which is equivalent to a chain of from 2 to 10 carbons, for example 4 to 9 carbons, more particularly 5 to 8 carbons, and especially 6 carbons.

In the linker radical -(Alk¹)ₙ-(X)ₘ-(Alk²)ₚ-, Alk¹ and Alk² when present independently represent an optionally substituted, straight or branched, C₁-C₆ alkylene, C₂-C₆ alkenylene or C₂-C₆ alkynylene radical. Presently it is preferred that any branching be modest, and indeed unbranched Alk¹ and Alk² radicals are currently most preferred. Similarly, although substitution is optional in Alk¹ and Alk², it is presently preferred that they be unsubstituted. Examples of Alk¹ and Alk² radicals include —CH₂—, —CH₂CH₂—, —CH₂CH₂CH₂—, —CH₂CH₂CH₂CH₂—, —CH═CH—, —CH═CHCH₂—, —CH₂CH═CH—, CH₂CH═CHCH₂—C≡C—, C≡CCH₂—, —CH₂C≡C—, and CH₂C≡CCH₂. Additional examples of Alk² include CH₂W—, —CH₂CH₂W— —CH₂CH₂WCH₂—, —CH₂CH₂WCH(CH₃)—, CH₂WCH₂CH₂—, —CH₂WCH₂CH₂WCH₂—, and —WCH₂CH₂— where W is —O—, —S—, —NH— or —N(CH₃)—.

In the linker radical -(Alk¹)ₙ-(X)ₘ-(Alk²)ₚ-, X when present represents an optionally substituted phenyl or 5- or 6-membered heteroaryl ring. Presently it is preferred that the ring X be unsubstituted. Examples of rings X include phenyl, pyridine, thiophene, and furan rings, but phenyl is presently preferred.

In the linker radical -(Alk¹)ₙ-(X)ₘ-(Alk²)ₚ-, n, m and p are independently 0 or 1, but since the linker radical must be present, at least one of n, m and p is 1. When m is 0, the linker radical is a hydrocarbon chain (optionally substituted and, depending on the identity of Alk², perhaps having an ether, thioether or amino linkage). When both n and p are 0, the linker radical is a divalent phenyl or heteraoaryl radical (optionally substituted). When m is 1 and at least one of n and p is 1, the linker radical is a divalent radical including a hydrocarbon chain or chains and a divalent phenyl or heteroaryl radical. In a particular subset of compounds of the invention the linker radical is an unsubstituted, unbranched, saturated hydrocarbon chain of from 4 to 9 carbons, more particularly 5 to 8 carbons, and especially 6 carbons.

In a preferred subset of compounds of the invention, R, has the formula -(Alk¹)ₙ-(X)ₘ-(Alk²)ₚ-Z wherein Alk¹, X, n and m are as defined in relation to formula (I), Z is —(C═O)NH(OH), p is 1 and Alk² is —CH₂—O—CH₂—, —CH₂—S—CH₂——CH₂—NH—CH₂—, —CH₂CH(OH)—, —CH₂CH(F)—, —CH₂C(F)₂—, or —CH₂(C═O)—.

The substituents R¹₂ and R₂, and R¹₃ and R₃

In the fused tetrahydropyridine ring of compounds (IA) and (IB), when R¹₂ is hydrogen R₂ may be any of the optional substituents listed above, such as trifluoromethyl, methyl, ethyl n- and iso-propyl, methoxy, ethoxy, methylenedioxy, ethylenedioxy, amino, mono- and di-methylamino, mono- and di-ethylamino, nitro, cyano, fluoro, chloro, bromo, and methylsulfonylamino.

Alternatively, when R¹₂ is hydrogen R₂ may a radical of formula -(Alk³)ᵣ-Q as defined above. In such radicals, r is 0 or 1; Alk³ may be, for example, —CH₂—, —CH₂CH₂— —CH₂CH₂CH₂—, —CH₂CH₂CH₂CH₂—, —CH═CH—, —CH═CHCH₂—, —CH₂CH═CH—, CH₂CH═CHCH₂—C≡C—, —C≡CCH₂—, —CH₂C≡C—, —CH₂C≡CCH₂— or —CH₂W—, —CH₂CH₂W— —CH₂CH₂WCH₂—, —CH₂WCH₂CH₂—, —CH₂WCH₂CH₂WCH₂—, and —WCH₂CH₂— where W is —O—, —S—, —NH— or —N(CH₃)—; and Q may be, for example, hydrogen or an optionally substituted phenyl, pyridyl, pyrimidinyl, thienyl, furanyl, cyclopropyl, cyclopentyl, cyclohexyl, piperidinyl, or morpholinyl. Presently Alk³ radicals which do not include ether, thioether or amino links, are preferred. Amongst rings Q which are presently preferred are phenyl, 4-pyridyl, and pyrimidin-2-yl. Optional substituents in rings Q may be selected from those listed above in the definition of the term “optionally substituted”. Examples of such substituents include trifluoromethyl, methoxy, methylenedioxy, ethylenedioxy, nitro, cyano, fluoro, chloro and bromo.

In a further alternative, R¹₂ and R₂ taken together with the carbon atoms to which they are attached may form an optionally substituted carbocyclic or heterocyclic ring, forming a spiro structure. Examples of such spiro-linked rings include cyclohexyl, piperidinyl spiro-linked at the 4-position, and pyrrolidinyl spiro-linked at the 2-position.

The above discussion of R¹₂, R₂ substituents applies also to R¹₃ and R₃.

The Substituent R₄

R₄ may be, for example, hydrogen, methyl, ethyl or n- or iso-propyl. Presently hydrogen is preferred.

The Fused Rings A¹ and A²

These rings are optionally substituted. Examples of optional substituents include trifluoromethyl, methyl, ethyl n- and iso-propyl, methoxy, ethoxy, methylenedioxy, ethylenedioxy, amino, mono- and di-methylamino, mono- and di-ethylamino, nitro, cyano, fluoro, chloro, bromo, and methylsulfonylamino.

Specific Examples of compounds for use in accordance with the invention include those of the Examples herein.

Hydroxamate compounds (IA) and (IB) of the invention may be prepared from the corresponding carboxylic acids, ie compounds (IA) and (IB) wherein in group R1 Z is —COOH by causing that acid or an activated derivative thereof to react with hydroxylamine, O-protected hydroxylamine, or an N,O-diprotected hydroxylamine, or a salt thereof, then removing the protecting groups from the resultant hydroxamic acid moiety (and from any protected substituents in the compound).

Conversion of the acid to an activated derivative such as the pentafluorophenyl, hydroxysuccinyl, or hydroxybenzotriazolyl ester may be effected by reaction with the appropriate alcohol in the presence of a dehydrating agent such as dicyclohexyl dicarbodiimide (DCC), N,N-dimethylaminopropyl-N′-ethyl carbodiimide (EDC), or 2-ethoxy-1-ethoxycarbonyl-1,2-dihydroquinoline (EEDQ).

Protecting groups for protection of reactive moieties in (II) during the reaction with hydroxylamine are well known per se, for example from the techniques of peptide chemistry. Amino groups are often protectable by benzyloxycarbonyl, t-butoxycarbonyl or acetyl groups, or in the form of a phthalimido group. Hydroxy groups are often protectable as readily cleavable ethers such as the t-butyl or benzyl ether, or as readily cleavable esters such as the acetate. Carboxy groups are often protectable as readily cleavable esters, such as the t-butyl or benzyl ester.

Examples of O-protected hydroxylamines for use in the above method include O-benzylhydroxylamine, O-4-methoxybenzylhydroxylamine, O-trimethylsilylhydroxylamine, and O-tert-butoxycarbonylhydroxylamine.

Examples of O,N-diprotected hydroxylamines for use in the above method include N,O-bis(benzyl)hydroxylamine, N,O-bis(4-methoxybenzyl) hydroxylamine, N-tert-butoxycarbonyl-O-tert-butyidimethylsilylhydroxylamine, N-tert-butoxycarbonyl-O-tetrahydropyranylhydroxylamine, and N,O-bis(tert-butoxycarbonyl)hydroxylamine.

Carboxylic acid analogues of compounds (IA) and (IB) may be prepared by coupling the tricyclic amine (IIA) or (IIB) with the carboxylic acid (III) or an activated derivative thereof 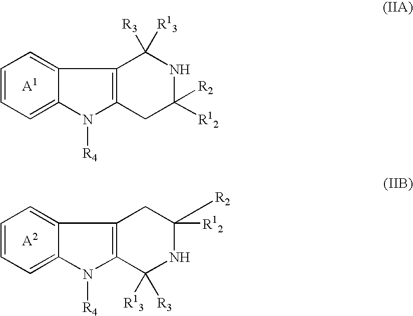
in which V is a protected carboxylic acid group, and thereafter removing the carboxy protecting group.

Condensation of the acid (III) with the amine (IIA) or (IIB) may be facilitated by dehydrating agents such as those referred to above.

In an alternative synthesis of compounds (IA) and (IB), a chlorotrityl-O—NH₂ resin (IV) may be reacted with an acid chloride (V) wherein —COOP is a protected carboxylic acid group, to produce a resin-supported protected carboxylic acid (VI).
Resin—ONH₂ (IV)
CICO—(Alk¹)ₙ-(X)ₘ-(Alk²)ₚ—COOP (V)
Resin—ONHCO—(Alk¹)ₙ-(X)ₘ-(Alk²)ₚ—COOP (VI)

The protecting group may then be removed from (VI) and the resultant acid coupled with the tricyclic amine (IIA) or (IIB) (analogously to the coupling of (IIA) or (IIB) and (IV) above). Finally the desired hydroxamate compound may be cleaved from the resin with trifluoroacetic acid.

N-hydroxyacylamino comounds of the invention may be prepared by coupling the tricyclic amine (IIA) or (IIB) with the carboxylic acid (VIII) or an activated derivative thereof. 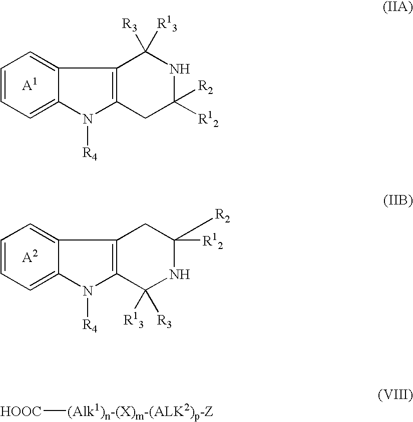
in which Z is halogen or other leaving group which is displaced with protected hydroxylamine. The resulting compound is then acylated with either an acid anhydride or acid chloride and the hydroxylamine protecting group removed to give the desired N-hydroxyacylamino compound.

Structures of formula (IIB) may also be prepared by the Pictet-Spengler reaction (1. Pictet, A; Spengler, T. Ber, 1911, 44, 2034; 2. Whaley, W. M.; Govindachari, T. R. Org. React., 1951, 6, 74.) which, in brief involves reaction of tryptamine or tryptophan or derivatives thereof and an aldehyde:

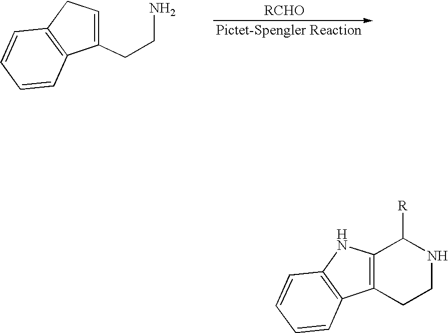

As mentioned above, the compounds with which the invention is concerned are HDAC inhibitors, and may therefore be of use in the treatment of cell proliferative disease, such as cancer, in humans and other mammals.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The following Examples illustrates the preparation of compounds of the invention. Their HDAC inhibitory properties are shown in Table 1 below. In the Examples, the following abbreviations have been used:
DMF: Dimethylformamide
MeOH: Methanol
DCM: Dichloromethane
TBME: t-Butylmethyl ether
PyBOP Benzotriazol-1-yloxotripyrrolidinophosphonium hexafluorophosphate
TFA: Trifluoroacetic acid

### EXAMPLE 1

Preparation of 8-Oxo-(1, 3, 4, 9-tetrahydro-β-carbolin-2-yl)-octanoic acid hydroxyamide 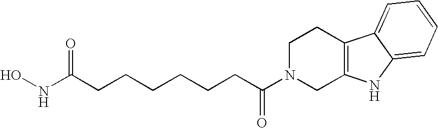
Stage 1—Immobilisation of linker with chlorotrityl-O—NH₂ resin

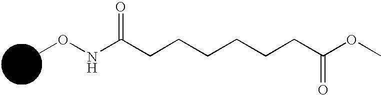

To a round bottomed flask charged with chlorotrityl-O—NH₂ resin (5 g, loading 1.36 mmol/g, 6.8 mmol) and DCM (50 ml) was added diisopropylethylamine (5.27 g, 40.8 mmol, 6 eq). Methyl 8-chloro-8-oxooctanoate (4.22 g, 20.4 mmol, 3 eq) was slowly added to the reaction mixture with orbital shaking and the reaction mixture shaken for 48 hours. The resin was filtered and washed, DMF, MeOH, DMF, MeOH, DCM, MeOH, DCM, MeOH×2, TBME×2. The resin was dried under vacuum.
Stage 2—Saponification

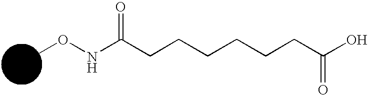

To a round bottomed flask charged with stage 1 resin (5 g, loading 1.36 mmol/g, 6.8 mmol) was added THF (17 ml) and MeOH (17 ml). To the reaction was added a solution of NaOH (1.36 g, 34 mmol, 5 eq) in water (17 ml). The reaction mixture shaken for 48 hours. The resin was filtered and washed with water×2, MeOH×2, DMF, MeOH, DMF, MeOH, DCM, MeOH, DCM, MeOH×2, TBME×2. The resin was dried under vacuum.
Stage 3—Coupling

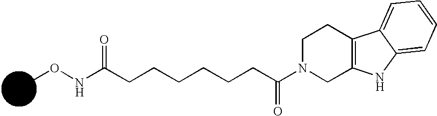

To a 2 ml 96 well plate charged with stage 2 resin (100 mg per well, loading 1.36 mmol/g, 0.136 mmol) was added a solution of PyBOP (0.21 g, 0.40 mmol, 3 eq) in DCM (0.5 ml) to each well. To one well was added 1,2,3,4-tetrahydro-9H-pyrido[3,4-B]indole (0.14 g, 0.82 mmol, 6 eq) in DCM (0.5 ml) followed by diisopropylethylamine (0.07 g, 0.54 mmol, 4 eq). The 96 well plate was sealed and shaken for 16 h. The resin filtered and washed, DMF, MeOH, DMF, MeOH, DCM, MeOH, DCM, MeOH×2, TBME×2.

Stage 4—Cleavage

A 2 ml Porvair plate was placed for collection under the 2 ml microlute plate from stage 3. A 2% solution of TFA/DCM (1.5 ml) was dripped through the resin in 0.5 ml aliquots, allowing 5 minutes between each aliquot. The procedure was repeated to give a total of 4 cleavage cycles. The solvent was removed using a Genevac. 8-Oxo-(1, 3, 4, 9-tetrahydro-β-carbolin-2-yl)-octanoic acid hydroxyamide (CHR-002504) was obtained as one product from the 96 reactions. 1H NMR (400 MHz, DMSO-d6) δ: 10.86 (1H), 10.34 (1H, s 8.67 (1H, s), 7.36 (1H, m, Ar), 7.27 (1H, m, Ar), 7.01 (1H, m, Ar), 6.95 (1H, m, Ar), 4.64 (2H, s, CH₂N), 3.75 (2H, m, CH₂), 2.72 and 2.63 (2H, m), 2.41 (2H, m), 2.17 and 1.91 (2H, m), 1.47 (4H, m), 1.26 (4H, m). m/z [ES] 344 [M+H]⁺

Further compounds of the invention may be prepared by methods analogous to those of Example 1 by using any of the tricyclic amines whose structures are shown in Tables 1A and 1B below and an acid chloride of formula
CH₃OOC-(Alk¹)ₙ-(X)ₘ-(Alk²)ₚ-COCl
(Alk¹, Alk², X, n, m and p being as defined in relation to formula (I) above) in place of 1,2,3,4-tetrahydro-9H-pyrido[3,4-B]indole and methyl 8-chloro-8-oxooctanoate of Example 1. The compounds of Examples 2, 3, 5, 6, and 8-14 to 17 of Table 1 below were prepared thus. The compounds of Examples 15-17 in Table 1 below were prepared by saponification of the corresponding methyl esters of Examples 11, 4 and 7, as follows:

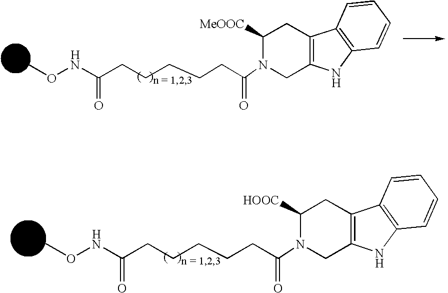

To a glass vial charged with resin (100 mg, loading 0.94 mmol/g, 0.094 mmol) was added a solution of NaOH (19 mg, 0.47 mmol, 5 eq) in H₂O (0.35 ml), THF (0.35 ml) and methanol (0.35 ml). The vial was capped and the reaction shaken for 48 h. The resin was filtered and washed with DMF, DCM, DMF, DCM, MeOH, DCM, MeOH×2, TBME×2. The resin was dried under vacuum. and activity versus HeLa Nuclear Extract HDACs as described above. The compounds of Examples 2 to 17 of Table 1 were characterised by mass spectrometry.

### Example 18

### N-Hydroxy-2-[5-oxo-5-(1,3,4,9-tetrahydro-beta-carbolin-2-yl)-pentyloxy]-acetamide

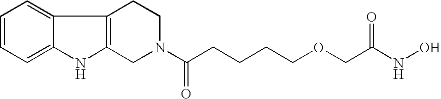 Reaction Scheme: 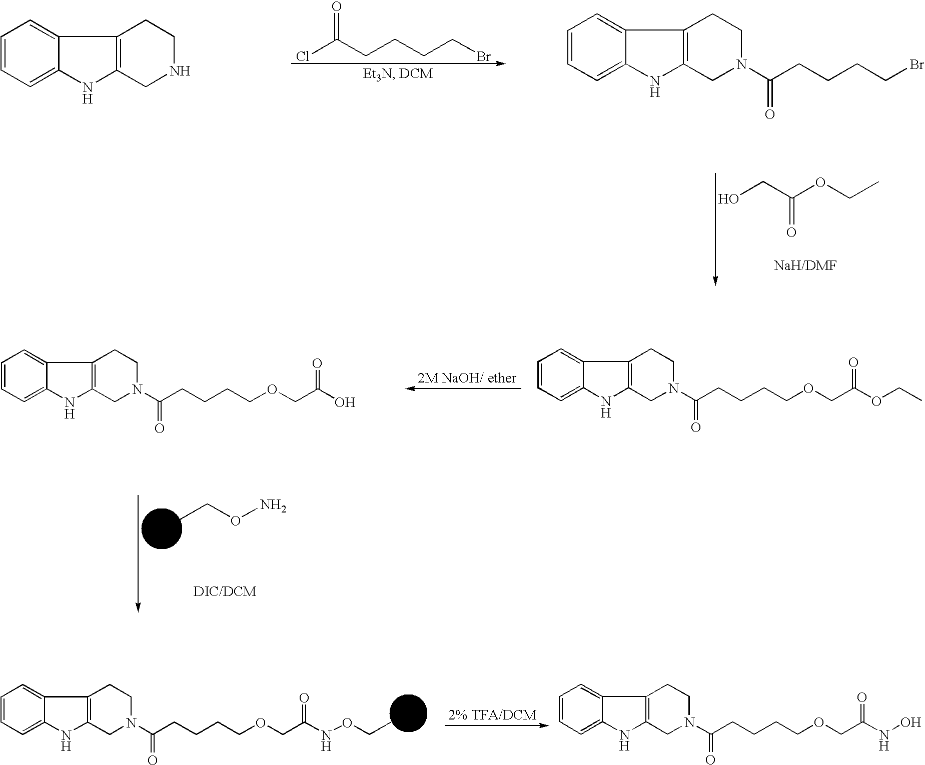
Stage 1

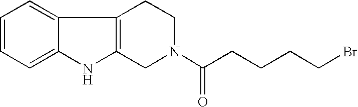

1,2,3,4-Tetrahydro-9H-pyrido(3,4-B)-indole (5 g, 29 mmol) in DCM (250 ml) was cooled to 0° C. 5-Bromovaleryl chloride (6.38 g, 32 mmol) was added dropwise. Triethylamine (4.5 ml, 32 mmol) was added and the reaction stirred at room temperature for 1.5 h. Sodium hydroxide (2M, 50 ml) was added and the reaction stirred for 10 minutes. The reaction mixture was diluted with water (50 ml). The organic phase was separated and the aqueous phase extracted with DCM. The combined organic phase was washed with acetic acid (5%), sodium bicarbonate (saturated) and water. The organic phase was dried (sodium sulphate), filtered and evaporated to dryness to give a crude solid. The solid product was gently swirled with DCM (50 ml) and quickly filtered. The required stage 1 product was obtained after filtration 4 g (65%) m/z 335 [M⁺+H]⁺, and was used in the next stage without further purification.
Stage 2

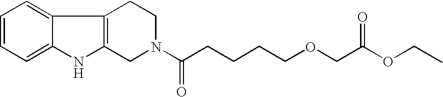

NaH (0.12 g, 2.98 mmol, 60% in mineral oil) was charged to a round bottomed flask under nitrogen. DMF (5 ml, anhydrous) was added and the slurry cooled to 0° C. Ethyl glycolate (0.28 g, 2.71 mmol) was added dropwise. The mixture was stirred for 2 hours at room temperature before cooling to 0° C. The bromo carboline stage 1 product (1 g, 2.98 mmol) was added dropwise in DMF (1 ml anhydrous) and the reaction stirred for a further 2 hr at room temperature. The reaction was acidified with NH₄Cl (saturated) and the reaction extracted with EtOAc (×3). The organic phase was dried (Na₂SO₄), filtered and the solvent removed in vacuo. The crude reaction mixture containing 50% product (LC-MS) was used in the next stage without further purification.
Stage 3

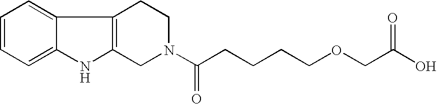

Crude carboline ester (1 g) from stage 2 was treated with NaOH (2M, 500 ml) and diethyl ether (500 ml). The reaction was stirred at room temperature for 1 hr. The reaction was acidified with (HCl, 2M). The aqueous layer was extracted with EtOAc (×3), dried (Na₂SO₄) and the solvent removed in vacuo. The crude carboline carboxylic acid (LC-MS purity 47%) was used in the next step without further purification.
Stage 4

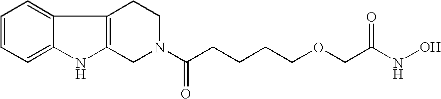

Hydroxylamine 2-chlorotrityl resin (296 mg, 1.14 mmol/g) was swollen in dichloromethane (7 ml). Crude carboline carboxylic acid (85 mg) from stage 3 was added to the reaction in DCM (2 ml). Diispropylcarbodiimide (98 mg) was added. The reaction was shaken for 0.5 hr. The resin was washed DCM, DMF (×2), DCM, MeOH (×2), MeOH, TBME before drying. The resin was cleaved with 2% TFA/DCM yielding 55.4 mg of crude product following solvent removal. The reaction was repeated using hydroxylamine 2-chlorotrityl resin (2.62 g, 1.14 mmol/g) and crude carboline carboxylic acid (760 mg) using the procedure described above. A crude yield of 445 mg was obtained. The combined crude material (500.4 mg) after resin cleavage was purified by prep-HPLC to give the required product (30 mg). m/z 346 [M⁺+H]⁺, 1H NMR (400 MHz, d4-MeOH) δ: 1.57-1.66 (4H, m, 2×CH₂), 2.50 (2H, m, CH₂), 2.6-2.75, (2H, m, CH₂), 3.43 (2H, m, CH₂), 3.78 (1H, m) 3.85 (3H, m, CH₂), 4.66 (2H, s, CH₂), 6.88 (1H, m, Ar), 6.95 (1H, m, Ar), 7.18 (1H, m, Ar), 7.3 (1H, m, Ar)

Measurement of Biological Activities

Histone Deacetylase Activity

The ability of compounds of Examples 1 to 17 to inhibit histone deacetylase activities was measured using the commercially available HDAC fluorescent activity assay from Biomol. In brief, the Fluor de Lys™ substrate, a lysine with an epsilon-amino acetylation, is incubated with the source of histone deacetylase activity (HeLa nuclear extract) in the presence or absence of inhibitor. Deacetylation of the substrate sensitises the substrate to Fluor de Lys™ developer, which generates a fluorophore. Thus, incubation of the substrate with a source of HDAC activity results in an increase in signal that is diminished in the presence of an HDAC inhibitor.

Data are expressed as a percentage of the control, measured in the absence of inhibitor, with background signal being subtracted from all samples, as follows:—
% activity=((Sⁱ−B)/(S^{o}−B))×100
where Sⁱ is the signal in the presence of substrate, enzyme and inhibitor, S^{o} is the signal in the presence of substrate, enzyme and the vehicle in which the inhibitor is dissolved, and B is the background signal measured in the absence of enzyme.

IC50 values were determined by non-linear regression analysis, after fitting the results of eight data points to the equation for sigmoidal dose response with variable slope (% activity against log concentration of compound), using Graphpad Prism software.

Histone deacetylase activity from crude nuclear extract derived from HeLa cells was used for screening. The preparation, purchased from 4C (Seneffe, Belgium), was prepared from HeLa cells harvested whilst in exponential growth phase. The nuclear extract is prepared according to Dignam J D 1983 Nucl. Acid. Res. 11, 1475-1489, snap frozen in liquid nitrogen and stored at −80° C. The final buffer composition was 20 mM Hepes, 100 mM KCl, 0.2 mM EDTA, 0.5 mM DTT, 0.2 mM PMSF and 20% (v/v) glycerol. IC50 results were allocated to one of 3 ranges as follows: Range A: IC50<330 nM, Range B: IC50 from 330 nM to 1000 nM; and Range C: IC50>1000 nM. Results are set forth in Table 1.

HeLa Cell Inhibition Assay

Some of the compounds of the Examples were tested for activity in the following assay:

Hela cells growing in log phase were harvested and seeded at 1000 cells/well (200 ul final volume) into 96-well tissue culture plates. Following 24 h of cell growth cells were treated with compounds (final concentration of 20 uM). Plates were then re-incubated for a further 72 h before a sulphorhodamine B (SRB) cell viability assay was conducted according to Skehan 1990 J Natl Canc Inst 82, 1107-1112.

Data were expressed as a percentage inhibition of the control, measured in the absence of inhibitor, as follows:—
% inhibition=100−((Sⁱ/S^{o})×100)
where Sⁱ is the signal in the presence of inhibitor and S^{o} is the signal in the presence of DMSO.

IC50 values were determined by non-linear regression analysis, after fitting the results of eight data points to the equation for sigmoidal dose response with variable slope (% activity against log concentration of compound), using Graphpad Prism software.

IC50 results were allocated to one of 3 ranges as follows: Range A: IC50≦1000 nM, Range B: IC50 from 1000 nM to 10,000 nM; and Range C: IC50>10,000 nM. Results are set forth in Table 1:

TABLE 1

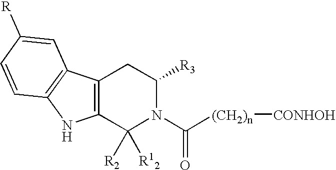

| | | | | | | | Inhibitor |
|---|---|---|---|---|---|---|---|
| | | | | | | | Activity |
| | | | | | | | versus |
| | | | | | | Inhibitor | Hela |
| | | | | | | Activity | Nuclear |
| | | | | | | versus | extract |
| Example | R | R₂, R¹₂ | R₃ | n | [M + H]+ | HDAC | HDACs |
| 1 | H | R₂ = H, | H | 6 | (NMR) | A | A |
| | | R¹₂ = H | | | | | |
| 2 | H | R₂ = H, | H | 5 | 330 | A | na |
| | | R¹₂ = H | | | | | |
| 3 | CH₃O— | R₂ = H, | H | 6 | 374 | A | na |
| | | R¹₂ = H | | | | | |
| 4 | H | R₂ = H, | CH₃OCO— | 6 | 402 | A | na |
| | | R¹₂ = H | | | | | |
| 5 | H | R₂ = H, | H | 7 | 358 | A | na |
| | | R¹₂ = H | | | | | |
| 6 | CH₃O— | R₂ = H, | H | 5 | 360 | A | B |
| | | R¹₂ = H | | | | | |
| 7 | CH₃O— | R₂ = H, | H | 7 | 388 | A | na |
| | | R¹₂ = H | | | | | |
| 8 | H | R₂ = H, | H | 5 | 398 | B | na |
| | | R¹₂ = CF₃ | | | | | |
| 9 | H | R₂ = H, | H | 6 | 412 | A | A |
| | | R¹₂ = CF₃ | | | | | |
| 10 | H | R₂ = H, | H | 7 | 426 | A | na |
| | | R¹₂ = CF₃ | | | | | |
| 11 | H | R₂ = H, | CH₃OCO— | 5 | 388 | B | na |
| | | R¹₂ = H | | | | | |
| 12 | H | R₂ = H, | CH₃OCO— | 7 | 416 | B | C |
| | | R¹₂ = H | | | | | |
| 13 | H | spiro | H | 5 | 398 | B | na |
| | | cyclohexyl | | | | | |
| 14 | H | spiro | H | 6 | 412 | A | B |
| | | cyclohexyl | | | | | |
| 15 | H | R₂ = H, | HOCO— | 5 | 374 | B | na |
| | | R¹₂ = H | | | | | |
| 16 | H | R₂ = H, | HOCO— | 6 | 388 | A | C |
| | | R¹₂ = H | | | | | |
| 17 | H | R₂ = H, | HOCO— | 7 | 402 | B | na |
| | | R¹₂ = H | | | | | |

Table 1A

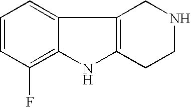

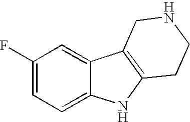

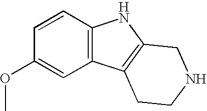

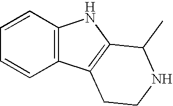

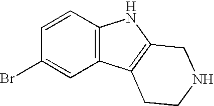

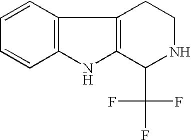

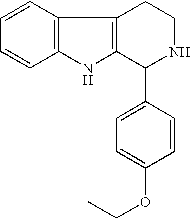

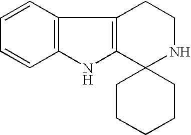

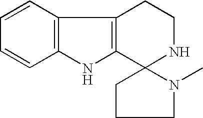

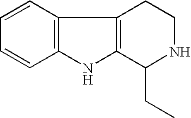

Table 1B

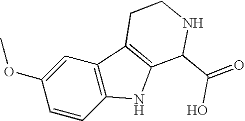

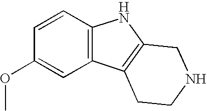

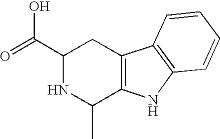

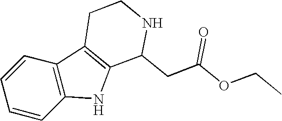

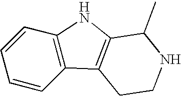

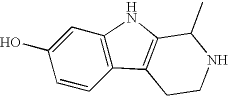

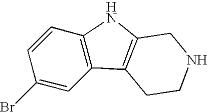

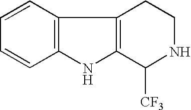

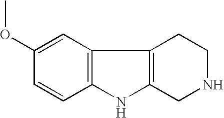

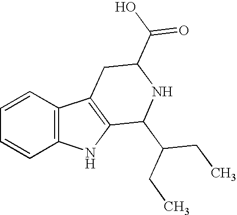

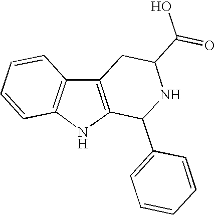

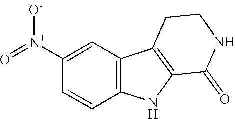

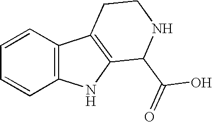

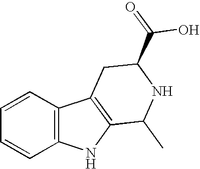

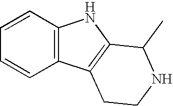

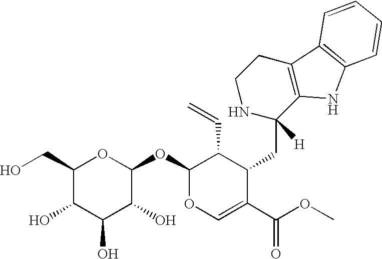

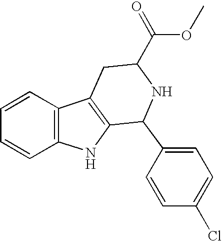

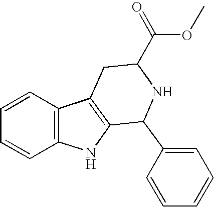

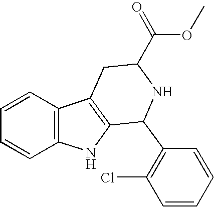

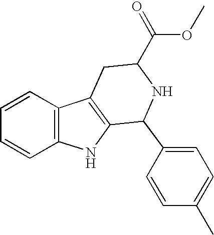

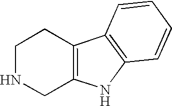

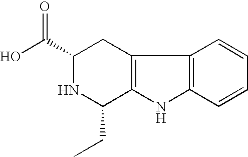

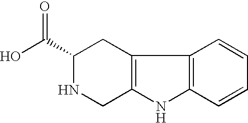

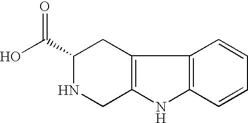

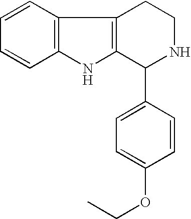

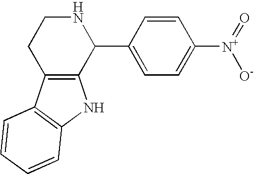

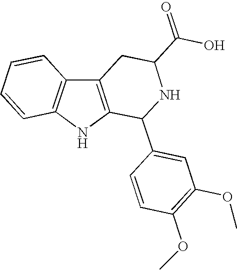

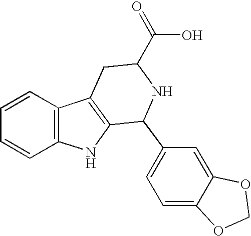

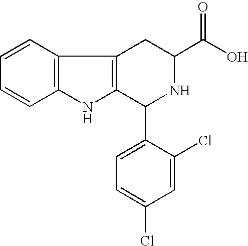

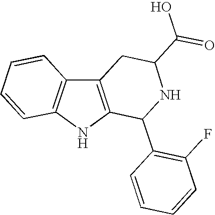

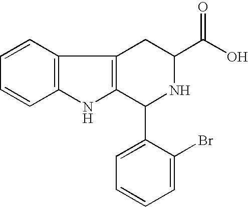

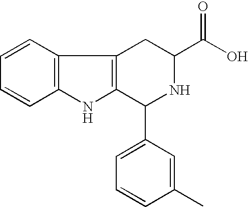

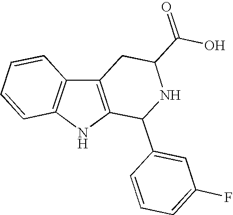

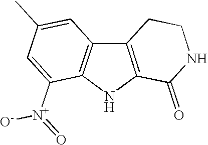

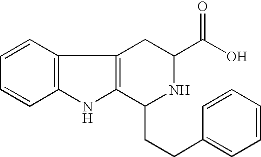

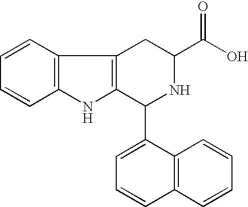

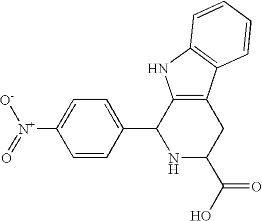

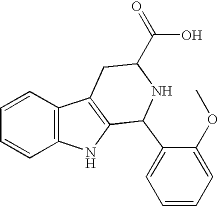

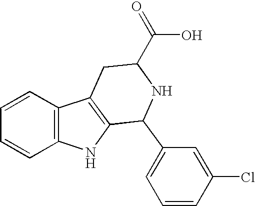

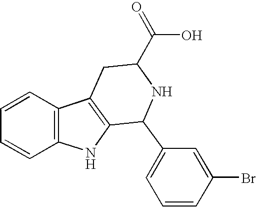

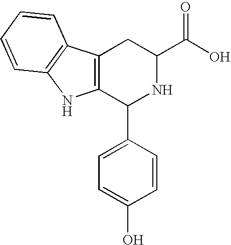

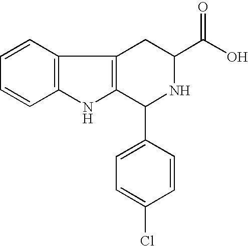

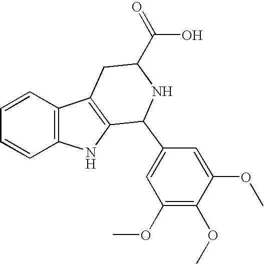

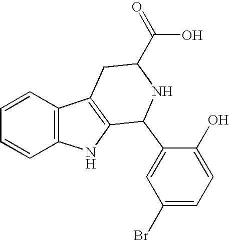

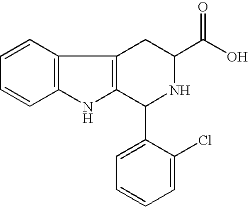

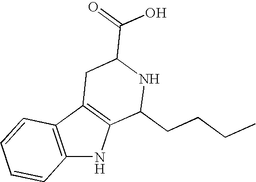

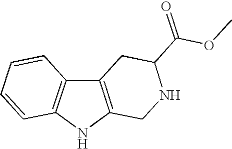

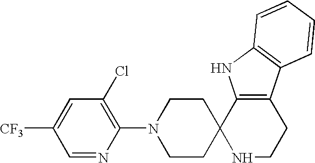

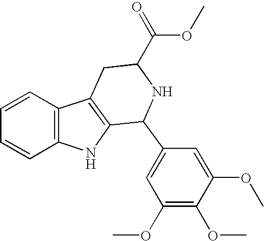

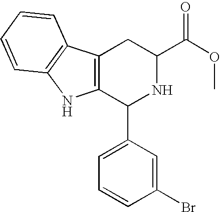

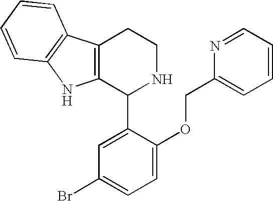

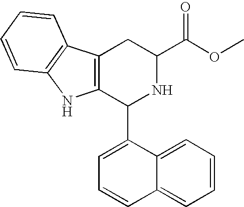

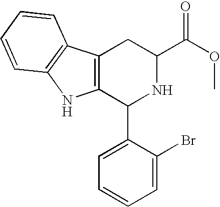

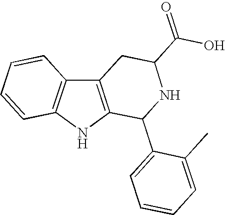

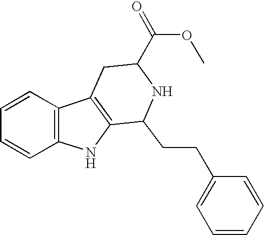

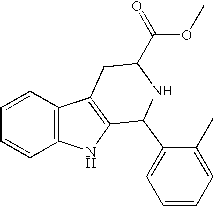

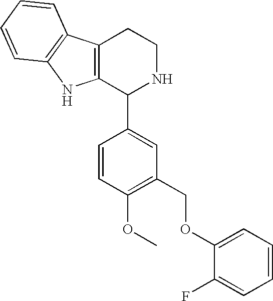

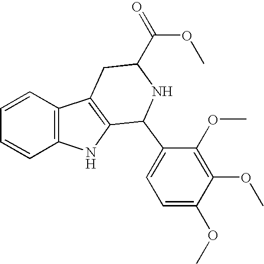

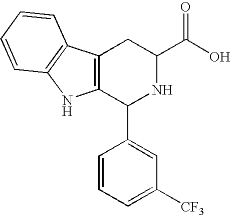

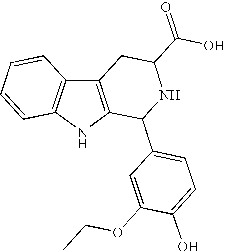

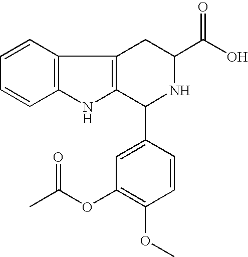

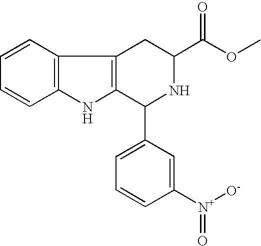

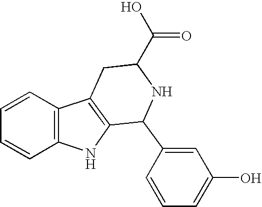

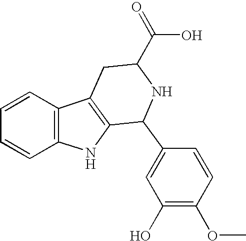

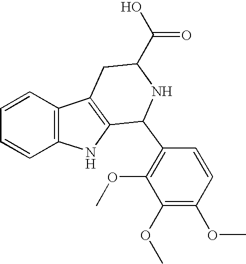

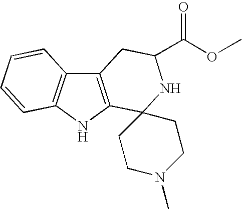

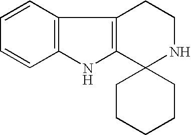

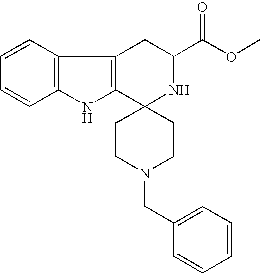

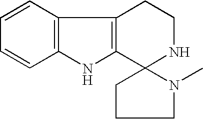

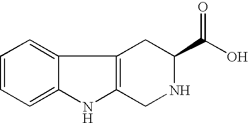

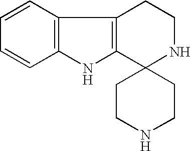

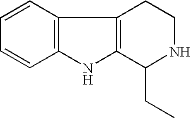

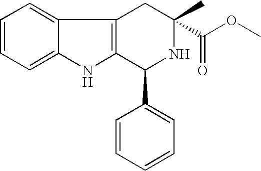

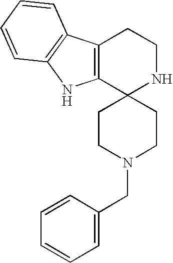

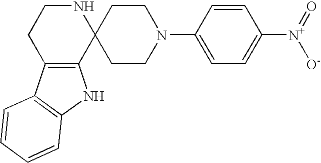

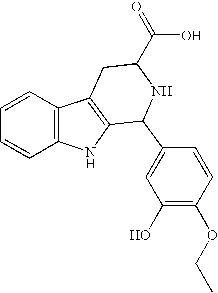

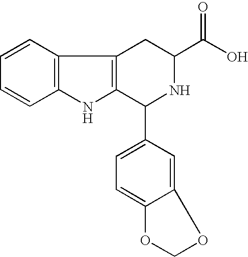

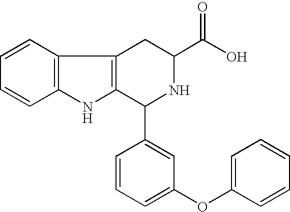

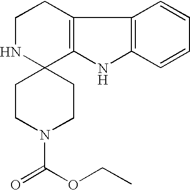

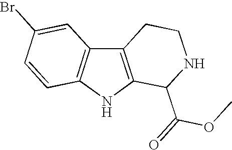

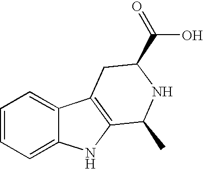

1. A compound of formula (IA) or (1B), or a salt, hydrate or solvate thereof 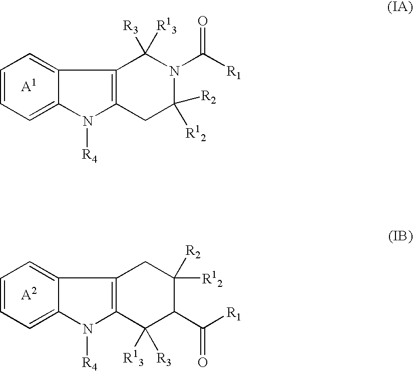
wherein fused rings A¹ and A² are optionally substituted;
R₁ represents a radical of formula -(Alk¹)ₙ-(X)ₘ-(Alk²)ₚ-Z wherein
Z represents a radical of formula —C(═O)NH(OH), or —N(OH)C(═O)Y wherein Y represents hydrogen, C₁-C₆ alkyl, a phenyl or cycloalkyl ring, or a monocyclic heterocyclic radical having 5 or 6 ring atoms;
Alk¹ represents an optionally substituted, straight or branched, C₁-C₆ alkylene radical,
Alk² represents an optionally substituted, straight or branched, C₁-C₆ alkylene, C₂-C₆ alkenylene or C₂-C₆ alkynylene radical which may optionally contain an ether (—O—), thioether (—S—) or amino (—NR^{A}—) link wherein R^{A} is hydrogen or C₁-C₃ alkyl;
X represents an optionally substituted phenyl or 5- or 6-membered heteroaryl ring; and
n, m and p are independently 0 or 1, provided that at least one of n, m and p is 1 and the length of radical -(Alk¹)ₙ-(X)ₘ-(Alk²)ₚ- is equivalent to that of a hydrocarbon chain of from 2-10 carbon atoms;
R¹₂ is hydrogen and R₂ is (a) an optional substituent or (b) a radical of formula -(Alk³)ᵣ-Q wherein r is 0 or 1, Alk³ represents an optionally substituted, straight or branched, C₁-C₆ alkylene, C₂-C₆ alkenylene or C₂-C₆ alkynylene radical and Q is hydrogen or an optionally substituted carbocyclic or heterocyclic group; or R¹₂ and R₂ taken together with the carbon atoms to which they are attached form an optionally substituted carbocyclic or heterocyclic ring;
R¹₃ is hydrogen and R₃ is (i) an optional substituent or (ii) a radical of formula -(Alk³)ᵣ-Q wherein r is 0 or 1, Alk³ represents an optionally substituted, straight or branched, C₁-C₆ alkylene, C₂-C₆ alkenylene or C₂-C₆ alkynylene radical and Q is hydrogen or an optionally substituted carbocyclic or heterocyclic group; or R¹₃ and R₃ taken together with the carbon atoms to which they are attached form an optionally substituted carbocyclic or heterocyclic ring; and
R₄ is hydrogen or C₁-C₆ alkyl.

2. A compound as claimed in claim 1 wherein the group Z in R₁ is a hydroxamate group —C(═O)NHOH or N-hydroxyformylamino group —N(OH)C(═O)H.

3. A compound as claimed in claim 1 wherein the length of the radical -(Alk¹)ₙ-(X)ₘ-(Alk²)ₚ- in R₁ is equivalent to a chain of from 2 to 10 carbons, or 4 to 9 carbons, or 5 to 8 carbons.

4. A compound as claimed in claim 1 wherein the length of the radical -(Alk¹)ₙ-(X)ₘ-(Alk²)ₚ- in R₁ is equivalent to a chain of 6 carbons.

5. A compound as claimed in claim 1 wherein, in radical R₁, Z is —(C═O)NH(OH), P is 1 and Alk² is —CH₂—O—CH₂—, —CH₂—S—CH₂—CH₂—NH—CH₂—, —CH₂CH(OH)—, —CH₂CH(F)—, —CH₂C(F)₂—, or —CH₂(C═O)—.

6. A compound as claimed in claim 1 wherein in the radical -(Alk¹)ₙ-(X)ₘ-(Alk²)ₚ-, Alk¹ and Alk² when present independently represent an unsubstituted, unbranched, C₁-C₆ alkylene, C₂-C₆ alkenylene or C₂-C₆ alkynylene radical.

7. A compound as claimed in claim 6 wherein in the radical -(Alk¹)ₙ-(X)ₘ-(Alk²)ₚ-, Alk¹ and Alk² when present independently represent —CH₂—, —CH₂CH₂—, —CH₂CH₂CH₂—, —CH₂CH₂CH₂CH₂—, —CH═CH—, —CH═CHCH₂—, —CH₂CH═CH—, CH₂CH═CHCH₂—, —C≡C—, —C≡CCH₂—, —CH₂C≡C— or —CH₂C≡CCH₂—.

8. A compound as claimed in claim 1 wherein, in the radical -(Alk¹)ₙ-(X)ₘ-(Alk²)ₚ-, X when present represents an unsubstituted phenyl ring.

9. A compound as claimed in claim 1 wherein the linker radical -(Alk¹)ₙ-(X)ₘ-(Alk²)ₚ-, m is 0 and n, p, or both are 1.

10. A compound as claimed in claim 1 wherein the linker radical -(Alk¹)ₙ-(X)ₘ-(Alk²)ₚ- is an unsubstituted, unbranched, saturated hydrocarbon chain of 4 to 9 carbons, or 5 to 8 carbons, or 6 carbons.

11. A compound as claimed in claim 1 wherein R¹₂ is hydrogen and R₂ is trifluoromethyl, methyl, ethyl, n- and iso-propyl, methoxy, ethoxy, methylenedioxy, ethylenedioxy, amino, mono- and di-methylamino, mono- and di-ethylamino, nitro, cyano, fluoro, chloro, bromo, or methylsulfonylamino.

12. A compound as claimed in claim 1 wherein R¹₂ is hydrogen and R₂ is a radical of formula -(Alk³)ᵣ-Q wherein r is 0 or 1; Alk³ is —CH₂—, —CH₂CH₂— —CH₂CH₂CH₂—, —CH₂CH₂CH₂CH₂—, —CH═CH—, —CH═CHCH₂—, —CH₂CH═CH—, CH₂CH═CHCH₂—; —C≡C—, —C≡CCH₂—, —CH₂C≡C—, —CH₂C≡CCH₂— or —CH₂W—, —CH₂CH₂W— —CH₂CH₂WCH₂—, —CH₂WCH₂CH₂—, —CH₂WCH₂CH₂WCH₂—, or —WCH₂CH₂— where W is —O—, —S—, —NH— or —N(CH₃)—; and Q is hydrogen or an optionally substituted phenyl, pyridyl, pyrimidinyl, thienyl, furanyl, cyclopropyl, cyclopentyl, cyclohexyl, piperidinyl, or morpholinyl.

13. A compound as claimed in claim 12 wherein Q is phenyl, 4-pyridyl, or pyrimidin-2-yl.

14. A compound as claimed in claim 1 wherein R¹₂ and R₂ taken together with the carbon atoms to which they are attached form an optionally substituted carbocyclic or heterocyclic ring.

15. A compound as claimed in claim 1 wherein R¹₃ is hydrogen and R₃ is trifluoromethyl, methyl, ethyl, n- or iso-propyl, methoxy, ethoxy, methylenedioxy, ethylenedioxy, amino, mono- and di-methylamino, mono- or di-ethylamino, nitro, cyano, fluoro, chloro, bromo, or methylsulfonylamino.

16. A compound as claimed in claim 1 wherein R¹₃ is hydrogen and R₃ is a radical of formula -(Alk³)ᵣ-Q wherein r is 0 or 1; Alk³ is —CH₂—, —CH₂CH₂— —CH₂CH₂CH₂—, —CH₂CH₂CH₂CH₂—, —CH═CH—, —CH═CHCH₂—, —CH₂CH═CH—, CH₂CH═CHCH₂—, —C≡C—, —C≡CCH₂—, —CH₂C≡C—, —CH₂C≡CCH₂— or —CH₂W—, —CH₂CH₂W— —CH₂CH₂WCH₂—, —CH₂WCH₂CH₂—, —CH₂WCH₂CH₂WCH₂—, or —WCH₂CH₂— where W is —O—, —S—, —NH— or —N(CH₃)—; and Q is hydrogen or an optionally substituted phenyl, pyridyl, pyrimidinyl, thienyl, furanyl, cyclopropyl, cyclopentyl, cyclohexyl, piperidinyl, or morpholinyl.

17. A compound as claimed in claim 16 wherein Q is phenyl, 4-pyridyl, or pyrimidin-2-yl.

18. A compound as claimed in claim 1 wherein R¹₃ and R₃ taken together with the carbon atoms to which they are attached form an optionally substituted carbocyclic or heterocyclic ring.

19. A compound as claimed in claim 1 wherein R₄ is hydrogen, methyl, ethyl or n- or iso-propyl.

20. A compound as claimed in claim 1 wherein optional substituents in the fused rings A¹ and A² are selected from trifluoromethyl, methyl, ethyl n- and iso-propyl, methoxy, ethoxy, methylenedioxy, ethylenedioxy, amino, mono- and di-methylamino, mono- and di-ethylamino, nitro, cyano, fluoro, chloro, bromo, and methylsulfonylamino.

21. A pharmaceutical composition comprising a compound as claimed in claim 1, together with a pharmaceutically acceptable carrier.

22. The composition of claim 21 containing an effective amount of the compound for inhibiting the activity of an HDAC enzyme

23. The composition of claim 22 wherein the activity is HDAC 1 activity.

24. The composition of claim 22 wherein the HDAC activity is ex vivo or in vivo.

25. (canceled)

26. (canceled)

27. A method for the treatment of a condition selected from the group consisting of cell-proliferation disease, polyglutamine disease, neurogenerative disease, autoimmune disease, organ transplant rejection, diabetes, haematological disorders and infection, which method comprises administering to a subject suffering such disease an effective amount of a compound as claimed in claim 1.

28. A method as claimed in claim 27 wherein the disease is cancer, Huntingdon disease, or Alzheimer disease.
